(19) United States
(12) Patent Application Publication
(10) Pub. No.: US 2011/0038904 A1
(43) Pub. Date: Feb. 17, 2011

(54) Gel Product

(75) Inventors: Charles J. Matteliano, Kenmore, NY (US); Barry A. May, Lockport, NY (US)

Correspondence Address: Kane Kessler P.C., 1350 Avenue of the Americas, 26th Floor, New York, NY, 10019, US

(73) Assignee: SILIPOS INC., Niagara Falls, NY (US)

(21) Appl. No.: 12/857,944

(22) Filed: Aug. 17, 2010

Related U.S. Application Data
(60) Provisional application No. 61/234,520, filed on Aug. 17, 2009.

(51) Int. Cl. A61K 8/02; A61K 9/00; A61Q 19/00; A61Q 5/12; A61P 17/00; A43B 13/38

(52) U.S. Cl. 424/401; 424/402; 424/400; 36/44

(57) ABSTRACT

A gel construct includes a viscoelastic gel pad with migratory oils or also synthetic or triglyceride fluids, emollients, waxes, vitamins, humectants, pharmaceutical actives, and other hair or skin conditioning or treatment agents. The gel construct also includes a body, hair or skin-side layer on one major side surface of the gel pad which is permeable to the migratory agents of the gel. The gel construct optionally includes a barrier layer on the opposite major side surface of the gel pad to prevent the migratory agents of the gel from penetrating that layer. The gel construct may find use as, for example, an insole.

### RELATED APPLICATIONS

This application is a nonprovisional application that claims the benefit of the filing date of U.S. Provisional Application No. 61/234,520, filed Aug. 17, 2009, the entire disclosure of which is incorporated by reference.

### BACKGROUND OF THE INVENTION

This invention relates to a gel product. Such a product can be used, for example, as a moisturizing pad and/or a medicating pad on a person's skin. Examples of products of this general type are shown in U.S. Pat. Nos. 6,117,119 and 6,673,054.

The gel component of these products is typically made from a composition including a polymer and a plasticizing oil or synthetic fluid. These gels are of the class often referred to as “crystal (or crystalline) gel”, “Block-Copolymer gel”, “visco-elastic gel”, or “TPE gel”. The gel component may alternatively include TPU gels, silicone Gels, hydrocolloid gels, hydro-gels, and other synthetic or natural elastomeric gels capable also of exuding various oils and synthetic or triglyceride fluids, emollients, waxes, vitamins, pharmaceutical actives, and other hair or skin conditioning or treatment agents.

In many of these gel products, the gel directly engages the skin of the wearer. An undesirable consequence of this is that the product may feel clammy to the touch. Also, friction of the skin on the gel can wear away the material of the gel.

### BRIEF DESCRIPTION OF THE DRAWINGS

Features of the invention will become apparent to one of ordinary skill in the art to which the invention pertains upon reading of the following description of an embodiment of the invention together with the accompanying drawings, in which:

FIG. 1 is a schematic perspective view of a gel product that is a first embodiment of the invention;

FIG. 2 is another schematic perspective view of the gel product of FIG. 1;

FIG. 3 illustrates use of the gel product as an insole for a shoe;

FIG. 4 is a fragmentary schematic sectional view through a gel product of the present invention;

FIG. 5A shows the two opposite sides of an insole made in accordance with the present invention, including both a body-side layer and a barrier layer on the gel pad;

FIG. 5B shows two more views of the insole of FIG. 5A;

FIG. 5C shows an insole made in accordance with the present invention, including a body-side layer but not a barrier layer on the gel pad, bonded to the inside of a sock, having been formed by compression molding; and

FIG. 5D shows additional views of the sock with insole of FIG. 5C.

### DETAILED DESCRIPTION OF AN EMBODIMENT OF THE INVENTION

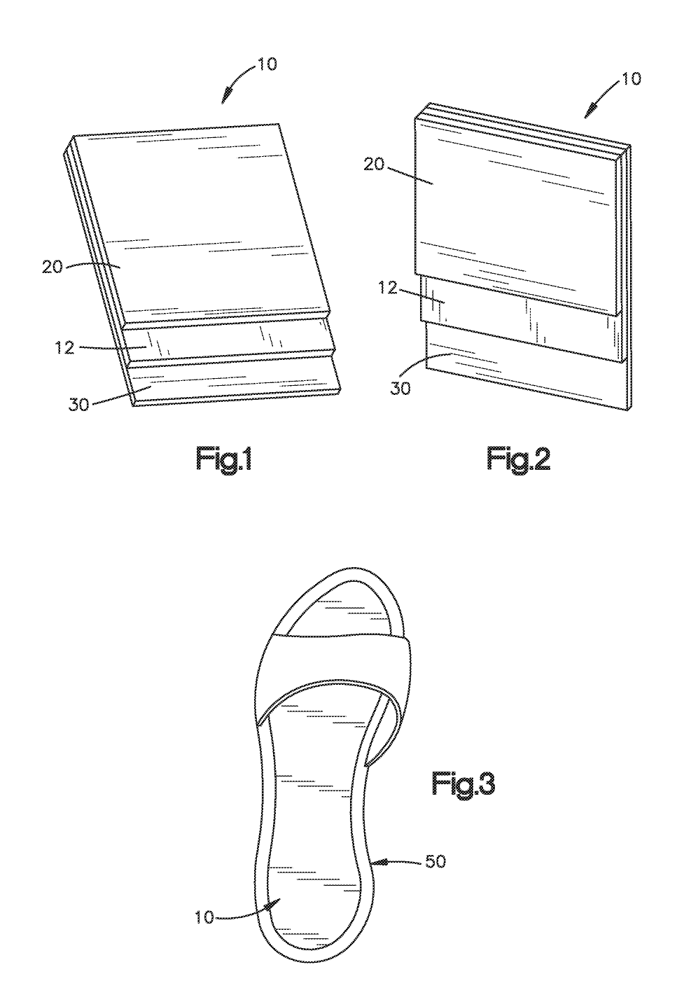

The present invention relates to a gel product. In particular, the present invention relates to a block-copolymer gel product for use as a therapeutic compound exuding pad for the skin, hair or body of a mammal. The therapeutic compound includes but is not limited to various oils and synthetic or triglyceride fluids, emollients, waxes, vitamins, pharmaceutical actives, and other hair or skin conditioning or treatment agents. The invention is applicable to gel products of various configurations such as laminates, multiple layer sandwiches, pillows, and composites. As representative of the invention, and without limiting the invention, FIGS. 1-4 illustrate a gel product 10 that is a first embodiment of the invention.

The gel product 10 (FIGS. 1-2) includes a gel pad 12; a treatment-side or body-side layer 20 on one side of the gel pad that is for engagement with the skin, hair or body of a mammal; and a barrier layer 30 on the opposite side of the gel pad. The barrier layer 30 is for prevention of exudation of the various oils and synthetic or triglyceride fluids, emollients, waxes, vitamins, pharmaceutical actives, and other hair or skin conditioning or treatment agents out of the gel pad beyond this layer and into any subsequent adhesive layer or other portion of any article or construct with which the product 10 is in contact.

The gel pad 12 may be made from any suitable gel as described herein and may be any viscoelastic gel which provides exudation of the various oils and synthetic or triglyceride fluids, emollients, waxes, vitamins, pharmaceutical actives, and other hair or skin conditioning or treatment agents that are suitable for the intended final application (customer use) of the finished product. In the illustrated preferred embodiment the gel pad 12 is made from a block copolymer gel. The primary components of such a gel are typically (though not always) a polymer and an oil, such as a block copolymer and a mineral, synthetic or triglyceride oil and other emollients and therapeutic ingredients or any combination thereof.

Suitable gels are described in, among others, U.S. Pat. Nos. 6,117,119 and 6,673,054, the entire disclosures of which are incorporated herein by reference. These materials can be formed into a gel pad which is simultaneously able to dissipate pressure and friction, absorb shock, and to exude various oils and synthetic or triglyceride fluids, emollients, waxes, vitamins, pharmaceutical actives, and other hair or skin conditioning or treatment agents to the skin, hair or body of a mammal.

Another suitable gel composition is described in commonly owned, pending U.S. patent application Ser. No. 12/557,496, filed Sep. 10, 2009, titled Gelatinous Elastomer Compositions, with named inventors: Charles J. Matteliano, Kenmore, N.Y.; Stephen P. Schaffer, Hamburg, N.Y.; and Stephen P. Sutton, Troy, N.C. The entire disclosure of this application is hereby incorporated by reference. This application describes a triglyceride gel that includes a migratory oil capable also of carrying other therapeutically beneficial ingredients to the skin, scalp, internal body cavity or hair of a human or other mammal. This product is commercially available, in various embodiments, from Silipos, Inc. as No. 06-087CS Triglyceride gel.

The body-side layer (top cover) 20 is made from a material (in this example a textile or fabric as described below but not limited to woven textile or fabric) that can be laminated to or otherwise bonded to or associated with the gel pad 12 and that has numerous small pores or other openings through which the oil from the gel can flow or can wick via capillary action.

The body-side layer 20 may function by capillary action to wick the oil through the body-side layer. Alternatively, the oil may simply flow through openings in the body-side layer, such as between fibers, rather than passing through the body-side layer by capillary action.

The size, number, and spacing of the pores, and the wicking capability (be it via construction or via other chemical wicking agent treatment) are considerations in providing an ultimate desired flow rate and any other flow characteristics. The flow rate is desirably quite small, only enough to moisturize adjacent skin, and not large enough to produce, for example, any “puddles” of oil.

The body-side layer 20 also acts to increase the strength (tensile, tear, abrasion) of the gel and prevent it from wearing out under the forces it is subjected to in this particular application (rubbing, shear, elongation strain and compressive strain).

The material from which the body-side layer 20 is made is selected for the following characteristics: oil flow characteristics; touch to the skin; durability; and other characteristics that make it suitable for the intended application, for example, an exposed layer of an insole for a shoe. The body-side layer 20 is preferably made from a woven or non-woven textile, or fabric, material but may also be any other construct which provides for the function of allowing the oils and synthetic or triglyceride fluids, emollients, waxes, vitamins, pharmaceutical actives, and other hair or skin conditioning or treatment agents to the skin, hair or body of a mammal to exude through the layer while providing for mechanical separation of the gel from direct contact to the skin, hair or body. Examples of such would be permeable urethanes, permeable plastic or polymer films, and other permeable materials which would provide similar mechanical advantages as a textile

One suitable commercially available textile is SX05001-196, which is available from Shinetex (Quanzhou) Co. LTD. This has the following specification: 50% polyester, 50% bamboo/charcoal mesh fabric (moisture management) 125 g/M2.

Other suitable textile materials are: All SX05001-XXX series textiles also available from Shinetex (Quanzhou) Co. LTD; Style #692650 100% Polyester PAT 243-FIN 2223 available from Milliken & Company; AKI Styl 3575 100% Polyester Knit Welt available from Adele Knits; Style #5720 92% Nylon-8% Spandex 2.8 oz./sq. yd. Mesh Fabric available from Metro Tex Sales, LLC.; and other textiles of construct or treatment suitable to the application.

A suitable non-textile material might be, for example, Spun Woven polyester or nylons or other synthetic resins and copolymers or Spun Woven TPE's and or TPU's.

The barrier layer 30 is bonded to the side of the gel pad 12 opposite the body-side layer 20. The barrier layer 30 serves several functions in the product 10. First, it provides a barrier to the oil, so that the oil from the gel pad 12 does not escape in a direction away from the body-side layer 20. The barrier layer 30 also provides an outer surface, on the finished product 10, to which an adhesive can easily and securely be bonded, so that the finished product can if desired be adhered to another item. The barrier layer 30 also provides a surface, more suitable than the gel, for contact with clothing, shoes, etc.

In the illustrated embodiment, the barrier layer 30 includes two components: a textile portion 36, and a barrier material portion 38 coated on the textile portion. The textile portion 36 of the barrier layer 30 does not need to be a wicking textile. One suitable material is a combination Adele Knits AKI Style 3575 100% Polyester (or other similar light to medium weight woven textile) which is subsequently coated or laminated with a thin layer of TPU commercially available from Fabrite Laminating Corporation.

The barrier material component 38 of the barrier layer 30 serves the following two primary functions: it provides an impervious barrier to the oil and it provides a suitable surface for receiving an adhesive on the opposite side of the gel while simultaneously allowing the gel to bond mechanically or chemically to the top of this layer.

The barrier material component 38 of the barrier layer 30, in this example, is a Thermoplastic polyurethane, though this component may also be any barrier material such as PET or other polymers, glass, ceramic, resin, rubber, silicone, or such material to prevent the various oils and synthetic or triglyceride fluids, emollients, waxes, vitamins, pharmaceutical actives, and other hair or skin conditioning or treatment agents to the skin, hair or body of a mammal from exuding through this barrier layer and to allow for bonding or assembly of the construct to some other surface (in the exemplary case a shoe bed).

A variety of suitable commercially available polyurethane formulations are available from Fabrite Laminating Corporation, Woodridge, N.J. Another suitable material for the barrier material component 38 of the barrier layer 30, other than polyurethane and PET, is: any other oil impervious coating such as Teflon, PVC, EVA, Viton, Polyester Films and Resins, and other polymers. Another suitable material for the barrier material component 38 is available from Catalyst, Inc. and is specified as “Deerfield PT6611 Polyether Urethane 1.0 mil coating thickness on Adele Knits Style 00855 100% Nylon Textile”.

The barrier layer 30 could have the barrier material component 38 (e.g., polyurethane) coated on only one side of the textile portion 36. Alternatively, the barrier layer 30 could have the barrier material component 38 (e.g., polyurethane) coated throughout (impregnated in) the textile portion 36. In the latter case, the barrier material component 38 itself should be able to bond to the gel 12. Many materials do not have this ability, because of the oil that exudes from the gel. Polyurethane alone does not have this ability to bond to the gel and requires the intermediate layer of textile or some other mechanical configuration (such as open cell foam structure, pores, loops, waffling, etc.) or alternately some other chemical treatment, to create a mechanical or chemical bond to both the polyurethane on one side and the gel on the other.

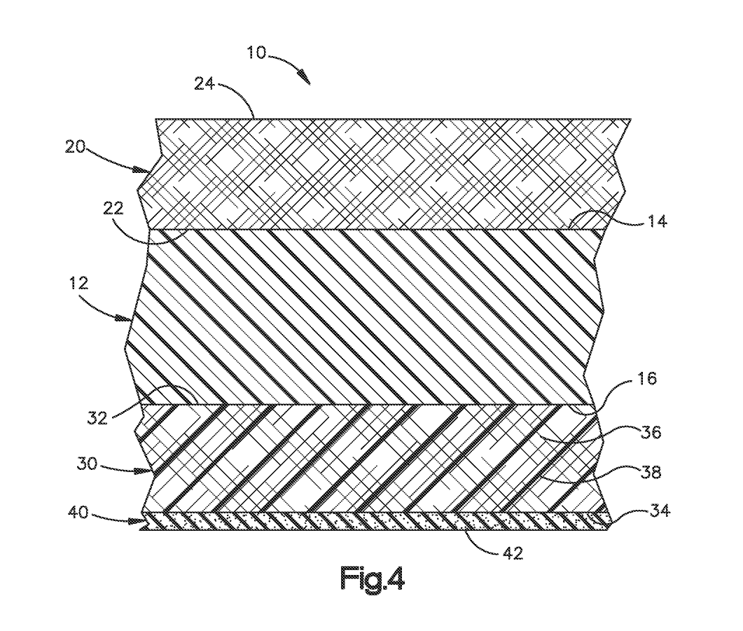

The final construct that is shown in the illustrated embodiment is produced by a process of laminating together the body-side layer 20, the gel pad 12, and the barrier layer 30. FIG. 4 is a schematic sectional view of a resulting gel product laminate 10. (FIG. 4 is, for clarity, not necessarily accurately representative of the relative thicknesses of the various layers. Also, the drawings symbols used to illustrate the various materials are schematic rather than representative.)

The final construct does not necessarily require lamination as a process. Other molding processes or assembly processes may also be used to produce the final construct. Examples of such would be simply to place the gel pad 12 between the body-side layer 20 and the barrier layer 30 and then sew the three layers together. Other methods could include bonding the gel layer 12 to either one of the other layers 20 or 30 to produce a similar construct. Co-extrusion of the various layers could also produce a similar construct. Other methods are discussed below.

The body-side layer 20 is preferably provided as a sheet material having opposite inner and outer major side surfaces 22 and 24. The body-side layer 20 may have a thickness in the range of from about 0.001 mm to 1.0 mm, but preferably in the range of about 0.05 to about 0.50 mm.

The gel 12 is also preferably provided as a sheet material (pad) of desired thickness, for example, in the range of from about 0.05 to 12.00 mm and more preferably in the range of about 1.0 to 6.0 mm. The gel pad 12 has opposite first and second major side surfaces 14 and 16.

The barrier layer 30 is preferably provided as a polyurethane-coated textile of desired thickness, for example, in the range of from about 0.001 mm to about 1.000 mm and more preferably in the range of about 0.02 mm to about 0.75 mm. The barrier layer 30 has opposite inner and outer major side surfaces 32 and 34.

In the laminating process of the illustrated embodiment, the inner major side surface 22 of the body-side layer 20 is bonded to the first major side surface 14 of the gel pad 12. The gel may impregnate partially (for example half way) into the body-side layer 20. The outer major side surface 24 of the body-side layer 20 is exposed in the finished laminate 10.

The inner major side surface 32 of the barrier layer 30 is bonded to the second major side surface 16 of the gel pad 12. The gel may impregnate partially (for example half way) into the barrier layer 30; this effect is more likely if the barrier layer 30 includes at least some textile portion that is not coated with polyurethane. The outer major side surface 34 of the barrier layer 30 is exposed in the finished laminate 10.

The gel product of the present invention can be used for many different purposes. These would include but are not limited to skin, hair or body treatment patches, wraps, pads, dressings, pressure relief pads, corn or callous removal pads or dressings, wart removal pads or dressings, cosmetic treatment pads or bandages and dressings, internal body cavity treatment devices, athletes foot or anti-microbial treatment insoles or pads or dressings or digital covers, open wound treatment dressings, burn dressings or pads which provide simultaneous pressure relief and treatment to the skin or body, moisturizing or anti-microbial cushioning bed-liners, or any other pad or cushion or dressing or wrap where an intermediate layer between the exuding gel and the skin, hair or body or internal body cavity or even teeth of a mammal is desired. A few additional specific examples are set forth herein and these examples are not limiting.

One preferred use of the gel product is as an insole or part thereof. An insole is the portion of footwear that is a thickness of material laid as an inner sole within a shoe, especially for comfort. The insole can be permanent (that is, adhered in the shoe), or it can be a slip-in (removable) piece. The product 10 with the adhesive 40 thereon can be cut to size, and placed in position in a shoe 50, as shown for example in FIG. 3. The adhesive 40 secures the product in position in the shoe 50.

If the gel product is used as a permanent insole, it may be prepared by bonding an optional layer of adhesive to the outer side surface of the polyurethane layer. FIG. 4 illustrates schematically the laminate 10 with an optional layer of adhesive 40 bonded to the outer major side surface 34 of the barrier layer 30. A release layer or strip (not shown) may, if appropriate, be provided on the exposed surface 42 of the adhesive 40. The polyurethane barrier material 34 advantageously provides a suitable surface for receiving an adhesive.

In this condition, the body-side layer 20 of the laminate 10 faces upward in the shoe, and its outer side surface 24 is engaged by the wearer's foot. Because the body-side layer 20 (which is preferably a textile or fabric material) is disposed between the wearer's foot and the gel pad 12, the wearer's foot is not in abutting engagement with the gel. Thus, the insole does not feel “clammy” to the touch. Nevertheless, the oil from the gel pad 12 can flow (or wick) through the body-side layer 20, as described above, to moisturize and/or medicate the wearer's foot.

An additional embodiment of a gel product of the present invention would include use of an anti-fungal or anti-microbial agent within the gel not only to moisturize and treat dry skin but also to treat or prevent other conditions of the foot such as athletes foot, fungal infections of the skin, microbial infections and ulcers associated with diabetes, etc.

The gel is a naturally exuding material which comprises a liquid portion mineral, triglyceride or synthetic oil or plasticizer and a solid fraction thermoplastic elastomer such as di-block and tri-block co-polymers. The oils can carry emollients, vitamins, humectants, and other therapeutic or pharmacologically active ingredients. The oil or plasticizer swells the polymer and together forms a cross-linked three dimensional elastomer gel network. The oil remains migratory and the rate of migration can be controlled with formulation and processing. The exudation requires contact with another surface which can accept the plasticizing oils and other ingredients via absorption or wicking. The wicking and flow characteristics of the body-side layer 20, in conjunction with the formulation of the gel, control the total exudation of oil from the gel pad 12. With the body-side layer 20 present on the surface 14 of the gel pad 12, oil is exuded from the gel, through the body-side layer, until a thin film of oil is present on the upper surface 24 of the body-side layer 20. At this point, the system reaches equilibrium, and no more oil exudes from the gel. When oil is thereafter removed from the upper surface 24 of the body-side layer 20, as by contact with the wearer's skin, more oil is exuded from the gel and flows through the body-side layer 20 to reestablish the thin film of oil on the upper surface of the body-side layer.

In this application, the barrier layer 30 is on the underside of the laminate 10 and thus faces away from the wearer's skin. It provides a barrier to the oil of the gel pad 12, so that the oil does not flow out of the gel pad in a direction away from the skin and toward the outsole of the shoe 50. This prevents oil from going onto the shoe, and also prevents oil from contacting and degrading the adhesive.

The barrier layer 30 is optional. Thus, in some applications, it may not be critical or important to block flow of oil out of the gel pad in a direction away from the body-side layer. In those cases, the barrier layer 30 can be omitted.

If the gel product is to be used as a removable insole, it can be cut to size and marketed in that form. No adhesive may be needed. If desired, the gel product can be further prepared by placing another finishing material on the surface which would be in engagement with the shoe.

One alternative use for a gel product of the present invention is as a moisturizing/cushioning bed liner. Other uses might include more targeted moisturizing/cushioning/pressure reduction applications to the foot, for example, for bunions, heel spurs, etc. Other uses might include moisturizing/cushioning/pressure reduction applications to the elbows or knees or bony prominences or distal ends of an amputated limb or on the outside of other human medical or animal veterinary device components whereby it is advantageous to not have direct contact of the gel to the body, mouth, internal body cavity or teeth.

Other applications for the product described herein would include, in addition to human use, veterinary use to provide for the same function to the body, hair, skin, or internal body cavity or teeth of a mammal. Examples of these uses would include a gel pad to be used inside a hoof wrap for treatment of hoof conditions and delivery of pharmacological treatment agents to an animal's hoof. In this case the body-side layer would provide for additional mechanical strength to the gel pad to prevent the pad from malformation caused by pressure of the hoof and yet still be able to deliver oils, emollients, humectants, pharmacological agents, anti-microbial agents (such as used to treat thrush in horses), or other treatment to the hoof and still provide padding for prevention of ulcerations and increased comfort to the hoof.

Other examples would include an animal skin or hide treatment patch or wrap whereby the wrap is of the illustrated configuration or is provided without the barrier layer 30 and is used to treat skin conditions on animals. Another example would be a pad of configuration 10 (FIGS. 1-2) used between an animal's back and a saddle, in which case the barrier layer 30 would prevent migration of the treatment agents from the gel pad 12 to the saddle, and the body-side layer would allow passage of the treatment agents to the skin of the animal to treat saddle sores. Yet another example would be a pad used between the cheek and gums of an animal to treat mouth sores and deliver pharmacological ingredients to the gum but not the cheek, or vice-versa, whereby the barrier layer would prevent migration of the treatment oils or pharmacological agents to one side or the other and the body-side layer would act to pass the pharmacological agents to the treatment zone required and provide for added mechanical and physical strength to prevent breakdown of the gel layer.

While the preferred embodiment is a laminate in which all layers are inherently bonded to each other, other constructs are possible. These could include, for example, a co-extrusion whereby layers are extruded together out of the same extrusion head and thereby bonded. Alternatively, the layers could simply be placed together without bonding but still in direct contact with each other (an “assembly”) to provide for the same function for each layer. The layers could alternatively be sewn together indirectly, for example like a pillowcase which has been stuffed with the gel layer and then sewn shut, or sewn together directly for example like a quilt whereby there would exist a top layer which would function as the body-side layer allowing for the various oils, humectants, treatment agents etc. to wick or pass through to the body and in addition a middle gel layer providing for the elastomeric cushioning and exudation of the various oils, humectants, treatment agents etc. and a barrier layer providing for prevention of migration of oils to that side all of which would be held together by cross-stitching or thread or some alternative attachment device or method for example such as textile rivets or staples. Alternatively both the bottom and top layers could be “body-side” layer materials allowing for the various oils, humectants, treatment agents etc. to wick through and also allowing the pad to be reversed and both sides of the pad to be used for “treatment”. Other processes by which these constructs could be produced include but are not limited to injection molding or dip molding or open pour molding or compression molding thereby bonding the gel layer onto either one or both of the top (body-side) layer and bottom (barrier) layer. Any of these constructs could then be further bonded or sewn into for example a shoe insole or onto some other garment to be worn to hold the pad in place on any portion of a mammal such as a human.

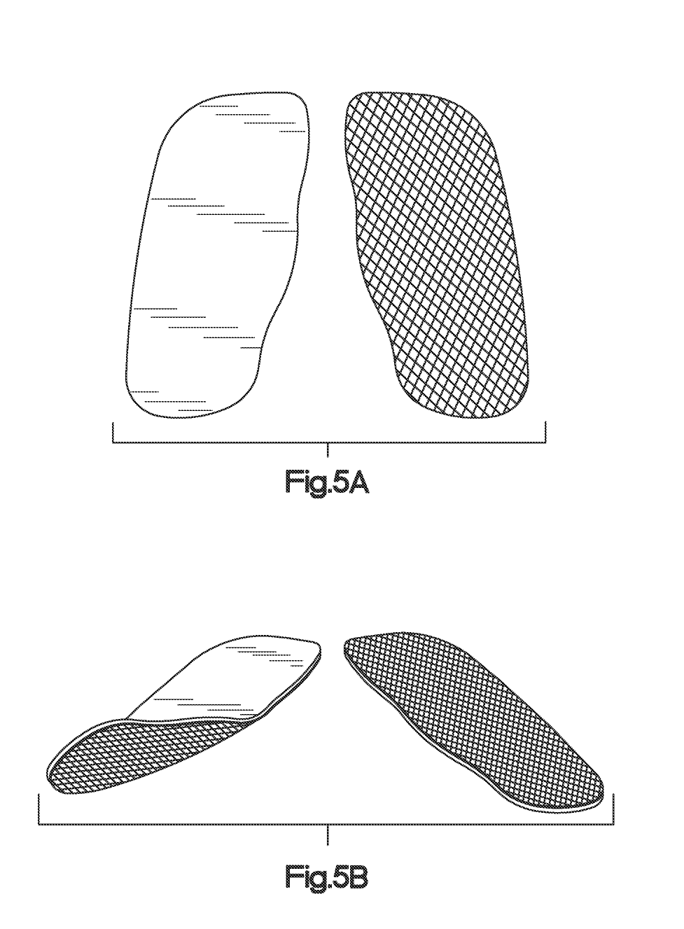

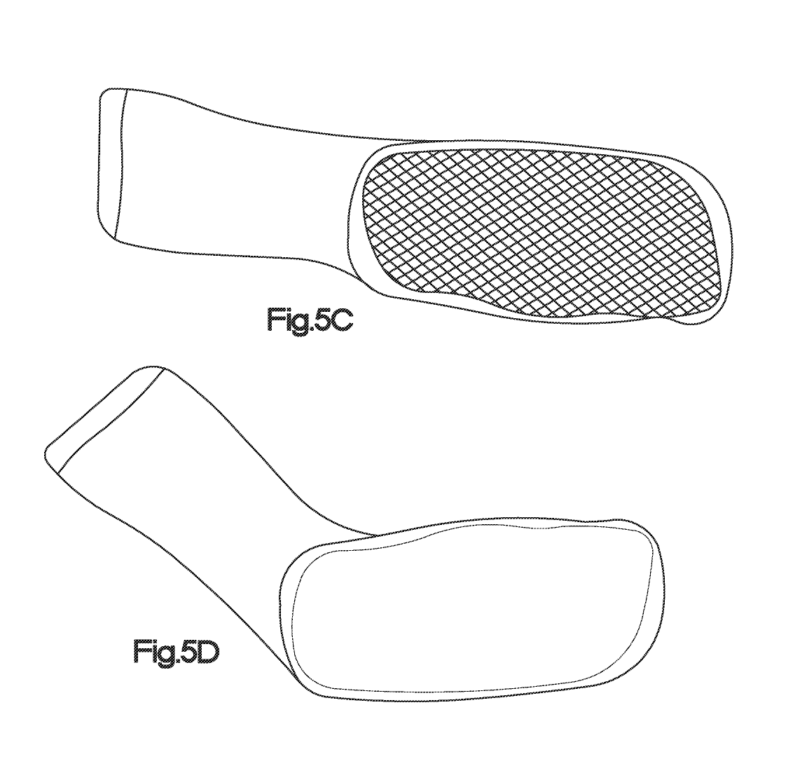

FIGS. 5A, 5B, 5C and 5D illustrate various ones of these embodiments of the present invention. FIG. 5A shows the two opposite sides of an insole made in accordance with the present invention, including both a body-side layer and a barrier layer on the gel pad. FIG. 5B shows two more views of the insole of FIG. 5A. FIG. 5C shows an insole made in accordance with the present invention, including a body-side layer but not a barrier layer on the gel pad, bonded to the inside of a sock, having been formed by compression molding. FIG. 5D shows additional views of the sock with insole of FIG. 5C. Other embodiments are possible.

1. A gel construct comprising:
a viscoelastic gel pad with migratory oils or also synthetic or triglyceride fluids, emollients, waxes, vitamins, humectants, pharmaceutical actives, and other hair or skin conditioning or treatment agents;
a body, hair or skin-side layer on one major side surface of the gel pad which is permeable to the migratory agents of the gel; and
a barrier layer on the opposite major side surface of the gel pad to prevent the migratory agents of the gel from penetrating that layer.

2. A gel product as set forth in claim 1 wherein the barrier layer comprises a textile portion and a polyurethane barrier material portion coated on the textile portion.

3. A gel construct comprising:
a viscoelastic gel pad with migratory oils or also synthetic or triglyceride fluids, emollients, waxes, vitamins, pharmaceutical actives, and other hair or skin conditioning or treatment agents; and
a body, hair or skin-side layer on major side surface of the gel pad which is permeable to the migratory agents of the gel.

4. A gel construct as set forth in claim 3 wherein the body-side layer has openings that allow flow of oil out of the gel pad through the body-side layer or is a wicking construct that allows capillary flow of oil out of the gel pad through the body-side layer.

5. A gel product comprising:
a gel pad made from a viscoelastic polymer gel with migratory oil;
a body-side layer on one major side surface of the gel pad that allows flow of oil out of the gel pad through the body-side layer; and
a barrier layer on the opposite major side surface of the gel pad that blocks flow of oil from the opposite major side surface of the gel pad.

6. A gel product as set forth in claim 5 wherein the barrier layer comprises polyurethane.

7. A gel product as set forth in claim 6 wherein the barrier layer comprises a textile portion and a polyurethane barrier material portion that is coated on the textile portion.

8. A gel product as set forth in claim 5 wherein the construct is made by a process of co-extrusion, lamination, sewing, stapling, riveting, molding, or bonding.

9. A gel product as set forth in claim 5 wherein the gel pad is a viscoelastic gel pad with migratory oils or also synthetic or triglyceride fluids, emollients, waxes, vitamins, humectants, pharmaceutical actives, and other hair or skin conditioning or treatment agents.

10. A gel product as set forth in claim 5 wherein the body-side layer has openings that allow flow of oil out of the gel pad through the body-side layer or is a wicking construct that allows capillary flow of oil out of the gel pad through the body-side layer.

11. A gel product comprising:
a gel pad made from a viscoelastic polymer gel with migratory oil; and
a body-side layer, bonded to one major side surface of the gel pad that allows flow of oil out of the gel pad through the body-side layer.

12. A gel product as set forth in claim 11 further including a barrier layer on the opposite major side surface of the gel pad that blocks flow of oil from the opposite major side surface of the gel pad.

13. A gel product as set forth in claim 11 wherein the gel pad is a viscoelastic gel pad with migratory oils or also synthetic or Triglyceride fluids, emollients, waxes, vitamins, humectants, pharmaceutical actives, and other hair or skin conditioning or treatment agents.

14. A gel product as set forth in claim 11 wherein the body-side layer has openings that allow flow of oil out of the gel pad through the body-side layer or is a wicking construct that allows capillary flow of oil out of the gel pad through the body-side layer.

15. An insole comprising:
a viscoelastic gel pad with migratory oils or also synthetic or triglyceride fluids, emollients, waxes, vitamins, humectants, pharmaceutical actives, and other hair or skin conditioning or treatment agents;
a body-side layer, bonded to one major side surface of the gel pad, with openings or wicking construct or wicking chemical treatment that allow flow of oil out of the gel pad through the body-side layer; and
a barrier layer on the opposite major side surface of the gel pad that blocks flow of oil from the opposite major side surface of the gel pad.
